(19) United States
(12) Patent Application Publication
(10) Pub. No.: US 2025/0034191 A1
(43) Pub. Date: Jan. 30, 2025

(54) LIGININ FLOWER PRODUCTION AND USES THEREOF

(71) Applicant: SILICYCLE INC., Québec (CA)

(72) Inventor: Georges THIBAUT KOUMBA, Québec (CA)

(21) Appl. No.: 18/704,749

(22) PCT Filed: Nov. 29, 2022
(86) PCT No.: PCT/CA2022/051743

§ 371 Date: Apr. 25, 2024

Related U.S. Application Data
(60) Provisional application No. 63/264,627, filed on Nov. 29, 2021.

(51) Int. Cl. C07G 1/00; A61K 8/33; A61K 8/35; A61K 8/368; A61K 8/9789; A61Q 19/00

(52) U.S. Cl. CPC C07G 1/00; A61K 8/33; A61K 8/35; A61K 8/368; A61K 8/9789; A61Q 19/00; A61K 2800/10

(57) ABSTRACT

A process for lignin depolymerization is disclosed herein. The process comprises providing a solution comprising dissolved lignin; adding an oxidation catalyst to the solution comprising dissolved lignin; and heating the solution to provide a depolymerized lignin product. Also disclosed is a depolymerized lignin product comprising at least one or more of the following: vanillin at 15-25 wt. %; apocynin at 6-12 wt. %; acetosyringone at 12-15%; syringaldehyde at 13-30 wt. %; syringic acid at 16-24 wt. %; vanillic acid at 5-15 wt. % for use as a cosmetic ingredient and/or food additive.

### CROSS-REFERENCE TO RELATED APPLICATION

The present application is claiming priority from U.S. Provisional Application No. 63/264,627 filed Nov. 21, 2021, the content of which is hereby incorporated by reference in its entirety.

### TECHNICAL FIELD

This disclosure relates to the deconstruction of lignin into its component monomers, and use of the monomers as cosmetic ingredients or food additives.

### BACKGROUND

In general, lignin can be used as a source of phenol, both in the formulation of adhesives for wood (US 2015/0329753 A1) and in the field of carbon fiber composite materials (U.S. Pat. No. 9,133,568 B2). Indeed, for lignins whose applications depend closely on their production technique, multiple applications may be concerned with composite materials as well as with cosmetics and food ingredients. The versatility of lignin is linked in particular to a demand for aromatic compounds that are likely to replace those obtained from fossil resources.

Regarding aromatic compounds intended for cosmetics and/or food processing, these are particularly abundant when deconstruction lignin. Indeed, phenols are generally produced after the depolymerization of lignin using chemical or biochemical catalysts. The depolymerization of lignin allows access to aromatic compounds such as cresols, catechols, guaiacols, vanillin and other phenolic monomers. These compounds are of great interest in the cosmetics and/or food industry. Indeed, 75% of the global demand for vanillin, considered to be the most used flavor in the food industry, is obtained from fossil sources such as crude oil.

In view of an ever-increasing demand for more “naturalness”, alternative sources for products including vanillin and derivatives thereof, such as apocynin (acetovanillone), are sought. Apocynin has been reported as an effective inhibitor of the NADPH-oxidase complex (Zhang, Yi et al., American Journal of Hypertension, 2005, 18 (7), 910-916).

Lignin is a highly heterogeneous polymer derived from a handful of precursor lignols that crosslink in diverse ways. The lignols that crosslink are of three main types, all derived from phenylpropane: coniferyl alcohol, sinapyl alcohol, and paracoumaryl alcohol. Lignin is one of the two major components of lignocellulose after cellulose. Lignin constitutes approximately 15-30 wt. % of lignocellulosic biomass and acts as a glue that fills the space between the other components binding them together to provide structural integrity. Lignin also plays a role in protecting against biological attacks and provides for water transport in the cell walls of a plant. Most available lignins are derived from black liquors, a by-product from the kraft process when digesting pulpwood into paper pulp removing lignin, hemicelluloses and other extractives from the wood to free the cellulose fibers. Depending on the process, three kinds of technical lignins are currently available in large quantities: kraft lignin (sulfate process), lignosulfonate (bisulfite process), and organosolv lignin (processing using organic solvent(s) to solubilize lignin and hemicellulose). The chemical structure of lignin is speculative and qualitative information can only be obtained through chemical and spectroscopic analysis. Several studies have reported on the oxidative breakdown of lignin in an alkaline medium or using a catalytic system such as CuSO₄ to yield simple phenolic compounds such as vanillin.

Unfortunately, the currently available methods for the deconstruction of lignin into its component monomers remain largely difficult and insufficient, often only providing access to a limited number of component monomers. As such, improved methods allowing for easier and more comprehensive access to such monomers are of commercial interest.

### SUMMARY

The present disclosure broadly relates to the deconstruction (“depolymerization”) of lignin into its component monomers. The present disclosure also relates to the use of such monomers as cosmetic ingredients and/or food additives.

In an aspect, the present disclosure relates to a process for the deconstruction or depolymerization of lignin. In an embodiment, the present disclosure relates to a process for the deconstruction or depolymerization of lignin into its component monomers. The present disclosure also relates to the use of such monomers as cosmetic ingredients and/or food ingredients.

In an aspect, the present disclosure relates to a deconstructed or depolymerized lignin, also referred to as lignin flower. In an embodiment of the present disclosure, the lignin flower comprises at least one or more of the following: vanillin (15-25 wt. %); apocynin (6-12 wt. %); acetosyringone (12-15%); syringaldehyde (13-30 wt. %); syringic acid (16-24 wt. %); and vanillic acid (5-15 wt. %).

In an aspect, the present disclosure relates to the use of lignin flower in cosmetic and/or food applications. In an embodiment of the present disclosure, the lignin flower is used in combination with a plant and/or three extract. In a further embodiment of the present disclosure, the combination may be for use as a cosmetic. In a further embodiment, the present disclosure relates to a combination comprising lignin flower and maple bark. In further embodiments, the maple bark comprises acertannin (ginnalin A).

In an aspect, the present disclosure relates to lignin flower rich in monophenols. In embodiments of the present disclosure, the monophenols comprise apocynin and vanillin. In an embodiment, lignin flower rich in apocynin may be formulated as a cosmetic composition for treating various skin conditions including effects associated with aging skin. In an embodiment, lignin flower rich in vanillin may be formulated as a food additive.

In an aspect, the present disclosure relates to a lignin monomer obtained from a deconstructed or depolymerized lignin. In embodiments, the present disclosure relates to the use of one or more lignin monomers obtained from the deconstructions or depolymerization of lignin as cosmetic ingredients and/or food additives.

It is provided a process for producing a depolymerized lignin comprising the steps of: providing a solution comprising dissolved lignin; adding an oxidation catalyst to the solution comprising dissolved lignin; and heating the solution to produce the depolymerized lignin.

In an embodiment, the solution comprising dissolved lignin further comprises an alkali.

In another embodiment, the alkali comprises sodium hydroxide.

In a further embodiment, the oxidation catalyst comprises sodium nitrite nanoparticles or carbon nanoparticles.

In an embodiment, the process described herein further comprises adding a co-catalyst to the solution comprising dissolved lignin.

In an embodiment, the co-catalyst comprises oxygen.

In a further embodiment, the pH of the solution being heated ranges from about 12 to about 14.

In an embodiment, the heating comprises temperatures ranging from about 180° C. to about 200° C.

In an embodiment, the process described herein further comprises adding an alcohol to the depolymerized lignin, filtrating said depolymerized lignin and purifying the filtrated depolymerized lignin.

In a further embodiment, the alcohol is isopropanol or ethanol.

In an embodiment, the depolymerized lignin product comprises at least one of vanillin at 15-25 wt. %; apocynin at 6-12 wt. %; acetosyringone at 12-15%; syringaldehyde at 13-30 wt. %; syringic acid at 16-24 wt. %; vanillic acid at 5-15 wt. %; and a combination thereof.

It is provided the use of a depolymerized lignin as encompassed herein in the manufacture of a cosmetic composition, a food additive, and/or an anti-inflammation composition.

It is also provided the use of a depolymerized lignin as encompassed herein as a cosmetic ingredient, a food additive, and/or an anti-inflammation ingredient.

It is further provided a cosmetic composition, a food additive, and/or an anti-inflammation composition comprising the depolymerized lignin produced by the process as described herein and a carrier.

The word “a” or “an” when used in conjunction with the term “comprising” in the claims and/or the specification may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used in this specification and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The deconstructed or depolymerized lignin may include a plurality of different lignin monomers. The distribution of the different lignin monomers may vary depending on the source material and process conditions used to effect deconstruction or depolymerization.

### BRIEF DESCRIPTION OF THE DRAWINGS

The following figures/drawings form part of the present specification and are included to further demonstrate certain aspects of the present specification.

FIG. 1A illustrates HPLC analysis of standard lignin flower.

FIG. 1B illustrates HPLC analysis following the deconstruction (depolymerization) of hardwood lignin with catalyst at 280 nm, in accordance with an embodiment.

FIG. 1C illustrates HPLC analysis following the deconstruction (depolymerization) of hardwood lignin without catalyst.

FIG. 2A illustrates an HPLC analysis identifying the different monomers resulting from oxidative depolymerisation of lignin flower from hardwood.

FIG. 2B illustrates an HPLC analysis identifying the different monomers resulting from oxidative depolymerisation of lignin flower from softwood.

FIG. 3 illustrates the dose-dependent cytotoxicity of pure lignin flower (A) and of a maple bark extract enriched with lignin flower (10 wt. %) (B), over a period of 24 hours, 48 hours and 6 days, in accordance with an embodiment of the present disclosure. The lignin flower exhibits substantially no cytotoxicity at all concentrations tested, whereas the maple bark extract enriched with lignin flower (10 wt. %) shows some cytotoxicity at higher concentrations (e.g., 25 and 50 μg/mL).

FIG. 4 illustrates the cytotoxicity on fibroblasts of the positive control (co-culture+10% SVF (stromal vascular fraction)); DMSO; and pure lignin flower at various concentrations (1.0, 2.5, and 5.0 μg/mL), in accordance with an embodiment of the present disclosure. The co-culture (biological medium; mature human adipocytes grown in 3D and human dermal fibroblasts cultured in 2D) alone is also depicted. Because fibroblasts play a crucial role in regulating skin physiology and cutaneous wound repair, and because of the intended use of the lignin flower as a cosmetic ingredient, it was important to test the cytotoxicity of the pure lignin flower on fibroblasts.

FIG. 5 illustrates the effects of adipocytes alone; the co-culture (biological medium; mature human adipocytes grown in 3D and human dermal fibroblasts cultured in 2D); DMSO; and sugar maple bark extracts enriched with pure lignin flower (10 wt. %), on adiponectin secretions by adipocytes, in accordance with an embodiment of the present disclosure. The sugar maple bark extract enriched with lignin flower (10 wt. %) was shown to increase adiponectin secretion in a dose dependent manner. Adiponectin regulates wound healing in the skin through promotion of proliferation and migration of keratinocytes, and also increases hyaluronic acid and collagen production. Adiponectin is also known to play a crucial role in the physiological metabolism of adipocytes, improving insulin sensitivity, and is known to exhibit anti-inflammatory and anti-fibrotic properties.

FIG. 6 illustrates the effects of the positive control (co-culture+10% SVF (stromal vascular fraction)); DMSO; the co-culture (biological medium; mature human adipocytes grown in 3D and human dermal fibroblasts cultured in 2D); and a maple bark extract comprising pure lignin flower (10 wt. %) at various concentrations (1.0, 2.5, and 5.0 μg/mL) on hyaluronic acid secretion, in accordance with an embodiment of the present disclosure. The maple bark extract comprising higher concentrations of lignin flower (5.0 μg/mL) was observed to significantly increase hyaluronic acid secretion. Hyaluronic acid, also known as hyaluronan, is a clear, gooey substance that is naturally produced by the body. The largest amounts of it are found in the skin, connective tissue and eyes. Its main function is to retain water to keep the tissues well lubricated and moist.

FIGS. 7A-B illustrate the effects of the co-culture (biological medium; mature human adipocytes grown in 3D and human dermal fibroblasts cultured in 2D); 10% SVF (stromal vascular fraction); and pure lignin flower at various concentrations (1.0, 2.5, and 5.0 g/mL), on the architecture of the extracellular matrix, in accordance with an embodiment of the present disclosure. As illustrated by two different markers, the lignin flower at the concentrations tested did not adversely affect the skin morphology.

FIG. 8 illustrates the effects of the positive control (co-culture+10% SVF (stromal vascular fraction)); the co-culture (biological medium; mature human adipocytes grown in 3D and human dermal fibroblasts cultured in 2D); DMSO; and maple bark extract enriched with lignin flower at various concentrations (1.0, 2.5, and 5.0 μg/mL) on collagen secretions, in accordance with an embodiment of the present disclosure. Collagen is the main structural protein in the extracellular matrix found in the body's various connective tissues; it is the main component of connective tissue and is mostly found in cartilage, bones, tendons, ligaments and the skin. The fibroblast is the most common cell that creates collage.

FIG. 9 illustrates a comparison between maple bark extract enriched with lignin flower and lignin flower alone on collagen secretions, in accordance with an embodiment of the present disclosure. The effects of the positive control (co-culture+10% SVF (stromal vascular fraction)); the co-culture (biological medium; mature human adipocytes grown in 3D and human dermal fibroblasts cultured in 2D).

FIG. 10 illustrates a 3D epidermis model grown in vitro (SkinEthic® RHE), in accordance with an embodiment of the present disclosure. The model illustrates that the pure lignin flower (10 wt. %) with maple bark extract of the present disclosure induces no mutagenic effect.

FIG. 11 illustrates the effects of lignin flower on anti-inflammatory activity from Griess test, in accordance with an embodiment of the present disclosure.

FIG. 12 illustrates the effects of lignin flower on procollagen secretions, in accordance with an embodiment of the present disclosure.

FIG. 13A illustrates the effects of coculture and dexamethasone as positive control on interleukin secretions, in accordance with an embodiment of the present disclosure.

FIG. 13B illustrates the effects of lignin at dose-dependant on interleukin secretions after three days, in accordance with an embodiment of the present disclosure.

FIG. 13C illustrates the effects of lignin at dose-dependant on interleukin secretions after six days, in accordance with an embodiment of the present disclosure.

FIG. 14 illustrate the confirmation of effect of the lignin extract on secretion of cytokine IL6.

### DETAILED DESCRIPTION

The present disclosure broadly relates to the deconstruction (“depolymerization”) of lignin into its component monomers. The present disclosure also relates to the use of such monomers as cosmetic ingredients and/or food additives. These and other aspects of the disclosure are described in greater detail below.

In the context of cosmetics, and in accordance with various embodiments of the present disclosure, one or more plant extracts may be combined with the lignin flower of the present disclosure. In an embodiment of the present disclosure, lignin flower may act as an immunomodulator. To that effect, low molecular weight lignin fragments have been previously described as having immunomodulatory properties (Kuroki T et al., Front. Microbiol. 2018, 9, 1164). Inflammation is part of the complex biological response of body tissues to harmful stimuli, such as pathogens, damaged cells, or irritants, and is a protective response involving immune cells, blood vessels and molecular mediators. Oral administration of certain anti-inflammatory drugs such as acetylsalicylic acid can sometimes be associated with risks of internal bleeding of vascular capillaries into surrounding tissues. In an embodiment, the lignin flower of the present disclosure may be combined with acetylsalicylic acid, at least due in part to its high apocynin content, known for its anti-inflammatory properties (Liu, N. et al., Nature, 2019, 568, 344-350). In a further embodiment, the present disclosure relates to the use of a composition comprising lignin flower rich in apocynin for use as a cosmetic composition for treating various skin conditions including effects associated with aging skin.

In embodiments wherein hardwood lignin is deconstructed under alkaline conditions without the use of a catalyst, the pH of the reaction mixture decreases from about 13 to about 9, with low amounts of monophenols (e.g., 0.7 wt. % vanillin) being obtained. Phenolic acids such as vanillic acid and syringic acid are substantially consumed. In embodiments using a catalyst, the pH decrease of the reaction mixture is significantly less pronounced, decreasing from about 13 to about 12, while also yielding higher amounts of monophenols (e.g., 4.9 wt. % vanillin).

In an embodiment, the depolymerized lignin product comprises at least one of vanillin at 15-25 wt. %; apocynin at 6-12 wt. %; acetosyringone at 12-15%; syringaldehyde at 13-30 wt. %; syringic acid at 16-24 wt. %; vanillic acid at 5-15 wt. %; and a combination thereof.

In particular, the depolymerized lignin product comprises at least one of vanillin at 20-25 wt. %; apocynin at 10-12 wt. %; acetosyringone at 12-15%; syringaldehyde at 13-16 wt. %; syringic acid at 16-20 wt. %; vanillic acid at 5-7 wt. %; and a combination thereof.

In the presence of a catalyst, the oxidation of the benzylic alcohol moieties of lignin was followed by further oxidation and bond cleavage. Substantial amounts of phenolic acids such as vanillic acid and syringic acid were detected. In embodiments of the present disclosure, the catalytic oxidation may be performed in the presence of molecular oxygen acting as a co-oxidant.

### EXAMPLES

### Example 1—Lignin Deconstruction (“Depolymerization”)-Lignin Flower

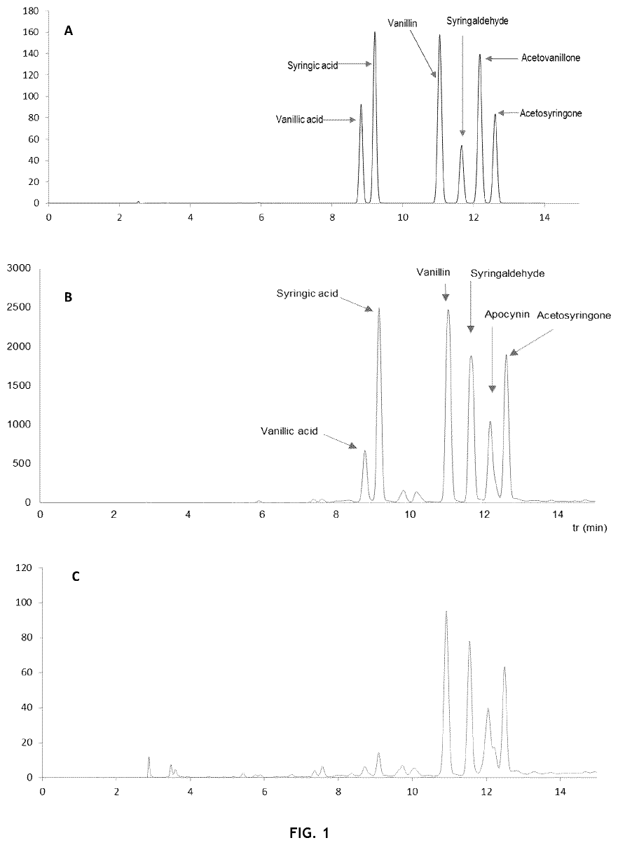

Hardwood organosolv lignin (100 g; Suzano Canada Inc.) is dissolved in a sodium hydroxide solution (500 mL; 2.0 M) at a pH ranging from about 12-13. A catalyst composed of sodium nitrite or carbon nanoparticles comprising grafted nitrite groups is subsequently added to the solution (up to 5.5 wt. % relative to the lignin weight). The reaction mixture is then pressurized using oxygen (10 bars) over a period of 30 minutes and then heated at 180° C. for 1.5 h. Following the deconstruction, the reaction mixture is cooled, and the pressure slowly released. Isopropanol (250 mL) is then added to the reaction mixture and the pH is lowered to about 2 using an aqueous (15%) sulfuric acid (H₂SO₄) solution. The resulting solution is stirred for a period of 1 h before decantation and filtration. Following filtration over a nylon membrane (1 μm), the isopropanol is evaporated. The solid residue was subsequently treated with isopropanol 20% in water and filtration over a nylon membrane (0.22 μm) and isopropanol removal before passing over a HP20 Diaion® resin to yield lignin flower. The lignin flower is subsequently extracted from Diaion resin by dissolving in EtOH and filtered. The lignin flower after ethanol evaporation and vacuum drying corresponds to 20-30 wt. % of the initial lignin mass. Different compounds, including acetovanillone, vanillin and other monomers, can subsequently be separated by column chromatography on silica (FIG. 1).

### Example 2—Maple Bark Extract Enriched with Lignin Flower

After grinding, bark particles (100 g) were extracted with an ethanol-water mixture (1:1, v/v; 1 L) at 85° C. over a period of 4 h. The extracted active ingredients were subsequently mixed with the lignin flower (10 wt. %). Following filtration, the mixture (maple bark extracts and lignin flower) was freeze dried and used without further treatment.

To validate the performance of lignin flower from hardwood, two types of lignin from different forest sources were tested as a model for extracting the lignin flower by oxidative depolymerization at 180° C. at basic pH and in the presence of sodium catalyst. Softwood lignin was obtained from pine while hardwood lignin was obtained from trembling aspen. The results are presented in Table 1.

TABLE 1
Comparative extraction between lignin
from hardwood and softwood

| | monomers | Polyphenols |
|---|---|---|
| Conditions | yield (%) | Quantification (GAE %) |
| Lignin depolymerization | 9.4 | 28 |
| from pine | | |
| lignin depolymerization | 22.8 | 45 |
| from aspen | | |

The different experimental conditions allowing access to the lignin flower clearly indicate that the percentage of depolymerization is more important for an extracted hardwood (yield of 22.8%) while that of a softwood leads to 9.4%. Similarly, the polyphenol content is higher for hardwood lignin than for softwood lignin (Table 1).

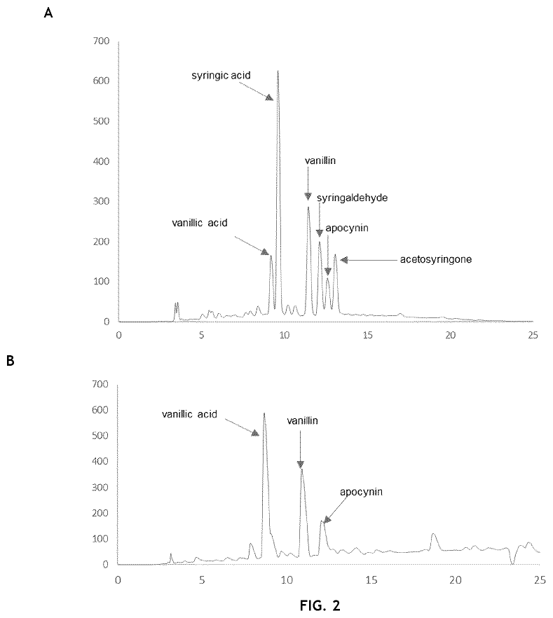

To visualize the differences between extracts from hardwood and softwood lignin, an HPLC method was developed to identify the different monomers resulting from oxidative depolymerization. All HPLC analyses were performed using an Agilent 1100 system including a G1311A pump, a G1322A degasser, a G1313A autosampler and a G1315B UV-DAD detector. A SiliaChrom Plus C8 column (5 μm, 4.6×250 mm) was used to identify the molecules by comparison of retention times of commercial standards. Mobile phase A was water with 0.1% formic acid and mobile phase B was acetonitrile also containing 0.1% formic acid. The elution gradient for phase A was 85% to 60% for the first 22 min, then 60% to 45% for 3 min, 45% to 40% for 2 min, 40% to 20% for 3 min and finally 20 to 85 for 2 min. The acquisition time was 32 minutes for a fixed flow rate of 0.75 mL/min. The injection volume was 3 μL and the detection wavelengths were 254 and 280 at 40° C. (see FIG. 2).

The comparison of HPLC profiles clearly indicates that extracts of lignin flower from hardwood are densely more abundant in terms of molecular population of monomer type than those derived of lignin flower from softwood. Indeed, in lignin flower from softwood, the guaiacyl moieties (vanillic acid, vanillin and apocynin) represent the largest proportion with 82% of the chromatogram peak area. Conversely, a proportion of 97% is measured for the lignin flower from hardwood for all monomers peak area. This represents a relatively pure proportion in terms of molecules of interest.

Cytotoxic Effect of Pure Lignin Flower and Sugar Maple Bark Extract Enriched with Lignin Flower (10 wt. %)

The cytotoxicity was determined using normal fibroblasts from human volunteers (MatTek #NHDF-CRY-AD) grown in a growth medium (MatTek #NHDF-GM). The cells were cultured at 37° C. with 5% CO₂. The cells were then seeded (2.5×10³ cells/well using a 96-well plate) and left to adhere overnight at 37° C., 5% CO₂. The next day, different concentrations of extract were added. A positive control, camptothecin (10 UM) was used. The cells were incubated for 24, 48 and 144 hours at 37° C., 5% CO₂. Four hours prior to the end of the incubation period, 20 μL/well of blue Alamar was added to the cell cultures. The latter is a non-toxic compound that contains resazurin, a non-fluorescent molecule that, in a living cell, is transformed into a fluorescent compound. The cellular activity is proportional to the number of living cells. After 4 hours of incubation with blue Alamar, the surnatant was transferred to a 96-well black plate and a fluorescence reading (EX560 nm/EM590 nm) was performed by spectrophotometry using a plate reader (Spark2, Tecan).

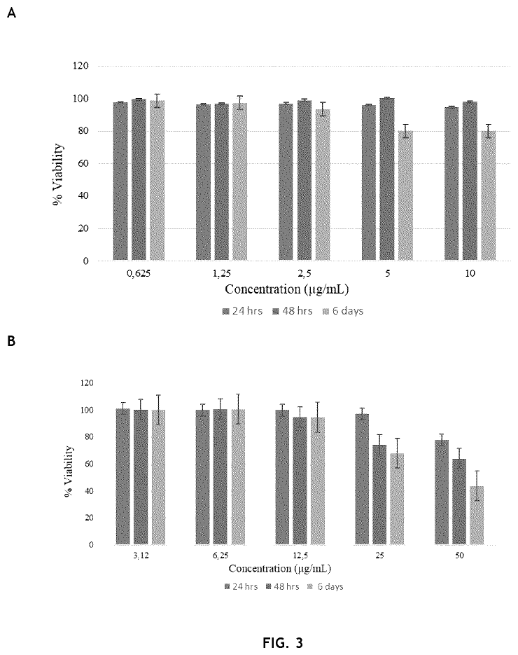

FIG. 3A shows the effect of different concentrations of lignin flower extract and shows that no loss of cell viability was observed at concentrations≤10 μg/mL after 24 and 48 hours of exposure. After 6 days (144 hours), however, there is a decrease of about 20% in cell viability at concentrations ≥5 μg/mL, still acceptable from a safety point of view. As such, lignin flower can be safely used at concentrations≤10 μg/mL. Moreover, the lignin flower, at all concentrations tested is not cytotoxic to skin fibroblasts. Maple bark extracts enriched with 10% lignin flower, show no loss of cell viability at concentrations≤12.5 μg/mL after 24 and 48 hours of exposure. However, some toxicity was observed at concentrations >than 25 μg/mL after 48 hours and 6 days of exposure (FIG. 3B). Maple bark extracts enriched with lignin flower (10 wt. %), at concentrations of 12.5 μg/mL; 6.25 μg/mL; and 3.12 μg/mL, are not cytotoxic to skin fibroblasts.

The cytotoxic effect of compositions comprising sugar maple bark extract enriched with lignin flower (10 wt. %) on fibroblasts was determined. The fibroblasts were obtained from a 56-year-old woman (BMI=26.8 kg/m²). Two assays of released lactate dehydrogenase (LDH) were performed on culture media (Day 1 and Day 7). The results were evaluated relative to the maximum cytotoxicity control (Triton-treated cells) and expressed as percentages: Percentage cytotoxicity=100×(Release of LDH under test conditions/LDH released under triton conditions). The cytotoxicity of the sugar maple bark extract enriched with lignin flower (10 wt. %) was evaluated on Day 3 and Day 6 on cocultures of 2D fibroblasts and 3D mature adipocytes.

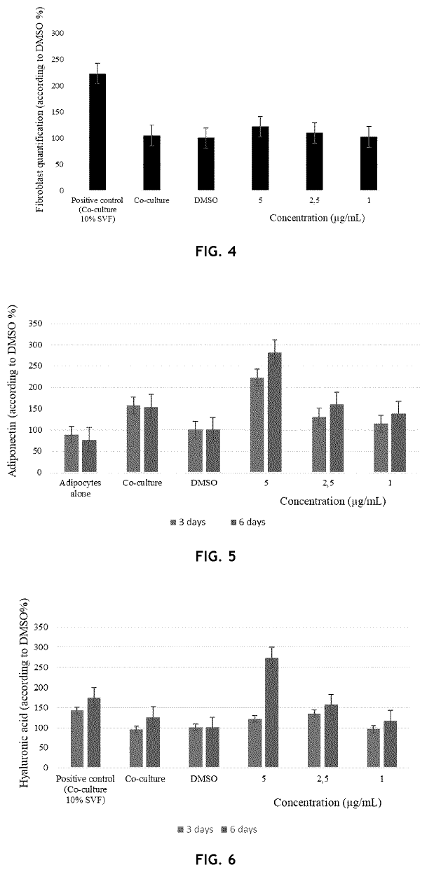

The results are expressed as a percentage relative to the coculture control conditions (FIG. 4). As expected, the SVF (10%) control exhibited a sharp increase in the number of fibroblasts (223%). DMSO was shown to have no effect on the number of fibroblasts on Day 6 of treatment relative to the coculture control. The sugar maple bark extract enriched with lignin flower (10 wt. %) was shown to increase the number of fibroblasts in a dose dependent manner while not being cytotoxic: 5.0 μg/mL-122%; 2.5 g/mL-110%; 1.0 μg/mL-102% (FIG. 5).

Effect of Sugar Maple Bark Extract Enriched with Lignin Flower (10 wt. %) on Adiponectin Secretions.

To further characterize the effects of sugar maple bark extracts enriched with lignin flower (10 wt. %), adiponectin secretion by adipocytes was evaluated in culture media on Day 3 and Day 6 following exposure (FIG. 6). Adiponectin secretion was increased when adipocytes are cocultured with fibroblasts (Day 3 and Day 6). Moreover, DMSO was observed to induce a decrease in adiponectin secretions relative to the coculture control (mature human adipocytes grown in 3D and human dermal fibroblasts cultured in 2D: co-culture AM3D-FB2D). The sugar maple bark extract enriched with lignin flower (10 wt. %) was shown to increase adiponectin secretion in a dose dependent manner. Day 3:5.0 μg/mL-222%; 2.5 μg/mL-130%; 1.0 μg/ml-114%. Day 6:5.0 μg/mL-281%; 2.5 μg/mL-159%; 1.0 μg/mL-137%.

Effect of Sugar Maple Bark Extract Enriched with Lignin Flower (10 wt. %) on Hyaluronic Acid (HA) Secretion.

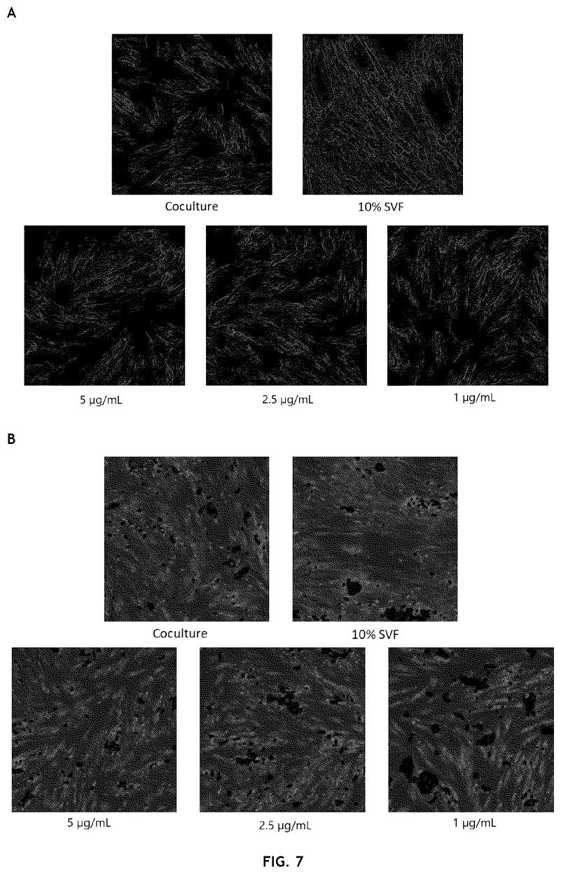

The effects of sugar maple bark extracts enriched with lignin flower (10 wt. %) on the extracellular matrix of fibroblasts was evaluated by measuring hyaluronic acid secretion by fibroblasts (FIG. 7). The positive control (co-culture+10% SVF) is used to highlight the increase in HA secretion relative to co-culture and solvent (DMSO) controls. Following the addition of SVF, an increase in HA secretion could be observed at Day 3 (142%) and Day 6 (174%). The solvent (DMSO) was shown to have no effect on HA secretions. Sugar maple bark extracts enriched with lignin flower (10 wt. %), at concentrations of 5.0 μg/mL and 2.5 μg/mL, increased HA secretions to 121% and 132% respectively (Day 3). After 6 days of treatment, the sugar maple bark extracts enriched with lignin flower (10 wt. %), at concentrations of 5.0 μg/mL, 2.5 μg/mL, and 1.0 μg/mL, increased HA secretions to 273%, 157% and 116% respectively.

Effect of Sugar Maple Bark Extract Enriched with Lignin Flower (10 wt. %) on the Architecture of the Extracellular Matrix.

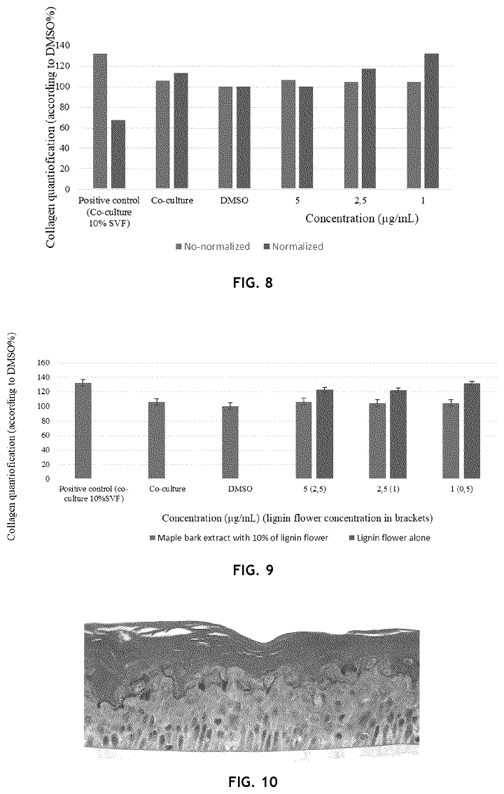

The effect of the sugar maple bark extracts enriched with lignin flower (10 wt. %) on fibronectin and collagen I was assessed. Fibroblasts were fixed at the end of the culture period, and fibronectin and collagen I proteins were labeled and measured by immunofluorescence (FIG. 8). For the immunofluorescence measurements, the fixed cells were blocked for 30 minutes in PBS 3% BSA (bovine serum albumin) and then incubated in the presence of primary antibodies to collagen 1 (Novusbio, NB600-408) and fibronectin (Novusbio, NBP2-22113) at a concentration of 1/100 overnight. After 3 washes with PBS saline buffer, the cells were further blocked for 30 minutes in PBS 3% BSA and further incubated in the presence of secondary antibodies at a concentration of 1/200 (Goat anti-rabbit Alexa-Fluor® 488 for collagen 1, ThermoFisher, A11008; Goat anti-mouse Alexa-Fluor® 568 for fibronectin, ThermoFisher, A11004) and DAPI (1/2500)). After 3 washes, the Collagen 1, Fibronectin and DAPI marked surfaces were quantified by image acquisition (4D microscopy technologies-3D+time) and processing (image processing and statistical analysis) (Imactiv 3D, Toulouse, France). The acquisition of seven microphotographs for each culture well was carried out using a fluorescence microscope (Cellnsight CX7 High Contente Screening, ThermoFisher). The labeling of fibronectin is, as expected, only fibrillary (FIG. 7A) while that of collagen I is not fibrillary but rather diffuse around the cells (FIG. 7B). A technical problem was observed with a deposition of fragments of the peptide hydrogel of 3D adipocytes on 2D fibroblasts. These deposits induced a strong autofluorescence in the same channel as the labeling of collagen I. In order to eliminate this bias, a maximum fluorescence threshold was set so as not to quantify these artifacts.

Regarding the collagen quantification, it can be observed that the trends are different between the results prior and after normalization (FIG. 8). After 6 days of treatment, and without normalizing, we have an upward trend in collagen production for maple bark extract at all three doses tested (5 μg/mL, 2.5 μg/mL and 1 μg/mL respectively). After normalization, the maple bark extract induces different effects depending on the dose tested with a relative no effect for 5 μg/mL (100%) and an upward trend with 2.5 μg/mL (116%) and 1 μg/mL (131%) (FIG. 9). It can be deduced that the maple bark extract, at all doses, tends to increase the production of Collagen 1 by its ability to increase the number of cells. Trends between normalized and none-normalized results for maple bark extract enriched with 10% of lignin flower appear to be related to the presence of lignin flower, for which it is inferred that the effect at the doses tested is related to an increase in the collagen production capacities of fibroblasts (FIG. 9).

This study evaluated the effects of a 6-day treatment with maple bark extract enriched with 10% of lignin flower at different concentrations on a coculture model of “aged” human adipocytes and skin fibroblasts to mimic the interactions between the hypodermis and the dermis in slightly aged human skin. This extract appears to have a dose-dependent proliferative effect on dermal fibroblasts, increasing their number after 6 days of treatment. In parallel, this product tends to increase the secretion of adiponectin by adipocytes in a dose-dependent manner. At the end of treatment, it tends to increase hyaluronic acid secretions.

This active ingredient (sugar maple bark extract enriched with 10% of lignin flower) therefore has potential anti-aging effects because it stimulates the proliferation of fibroblasts while maintaining the metabolism of adipocytes and could limit skin inflammation that develops over time. In addition, it would help to improve the extracellular matrix and maintain the hydration of the skin by stimulating the secretions of hyaluronic acid by fibroblasts. Indeed, the presence of the lignin flower in the bark extract, would promote the anti-aging effect through its properties of inhibition of the secretion of the pro-inflammatory cytokine IL-6 and a potential stimulatory effect of collagen production. Maple bark extracts enriched with 10% lignin flower at tested doses (5/2.5/1 μg/mL) was not cytotoxic for coculture and promotes secretions of hyaluronic acid and collagen.

### Antioxidant Activity

An evaluation of the anti-free radical activity of the two types of lignin was carried out with DPPH test and the results are shown in Table 2.

TABLE 2
Test DPPH

| | Polyphenol | | |
|---|---|---|---|
| | content | Antioxidant activity (DPPH) | |
| Lignin flower | (GAE %) | IC₅₀ (μg/ml) | Trolox Eq |
| From softwood (pine) | 28 | 23.4 | 5.1 |
| From hardwood (aspen) | 45 | 7.7 | 1.7 |

Compared to the softwood lignin extract which presents a polyphenol content of 28% in gallic acid equivalent, the hardwood lignin extracts leads to a higher polyphenol content with 45%. In terms of antioxidant activity, it is the lignin flower from hardwood that presents the best antioxidant profile with an IC₅₀ of 7.7 μg/mL against 23.4 μg/mL for lignin from softwood. This antioxidant activity of the lignin flower from hardwood shows a more marked effectiveness in particular for extracts rich in syringic polyphenols.

### Anti-Inflammatory Activity: (Griess Test)

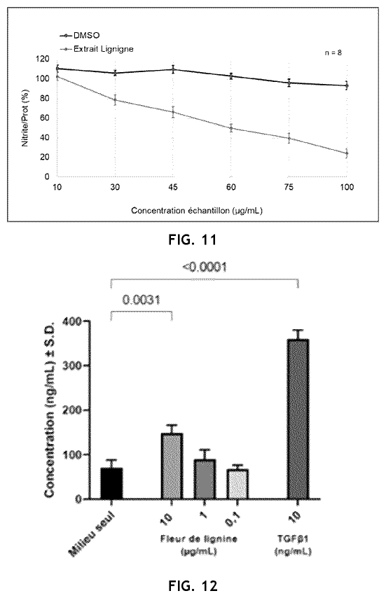

Lignin extract decreases nitrite production from 30 μg/mL to 100 μg/mL (n=8) (see FIG. 11). This result suggests an important capacity of the lignin flower (from hardwood) to limit or correct any inflammatory state (decrease of nitrites up to 75%).

### Procollagen Stimulation

The lignin flower extract (hardwood) was diluted in DMSO to a concentration of 10 mg/mL. The extract was subsequently diluted in the culture medium to obtain the final concentrations needed for the study. The final concentration of DMSO in the culture medium was 0.1%. According to a previous cytotoxicity study, a concentration up to 10 μg/mL did not influence cell viability. This concentration was therefore selected as a starting point. In addition to the 10 μg/mL concentration, two other concentrations of the extract (0.1 and 1 μg/mL) were used. As a positive control, recombinant human TGFβ1 (Peprotech) was used. This was diluted in the culture medium to obtain a final concentration of 10 ng/ml.

Different concentrations of lignin flower extract were used (see FIG. 12). A dose-response effect is noted. In addition, the concentration of 10 μg/mL induces significant procollagen production compared with cells incubated with the medium alone. Thus, this extract contains components capable of inducing collagen production. The lignin flower extract clearly induces a significant production of procollagen in human dermal fibroblasts compared to cells not exposed to the extract. The assay was performed on fibroblasts from a single donor.

### Test Element on Interleukin 6 (IL6) Secretions

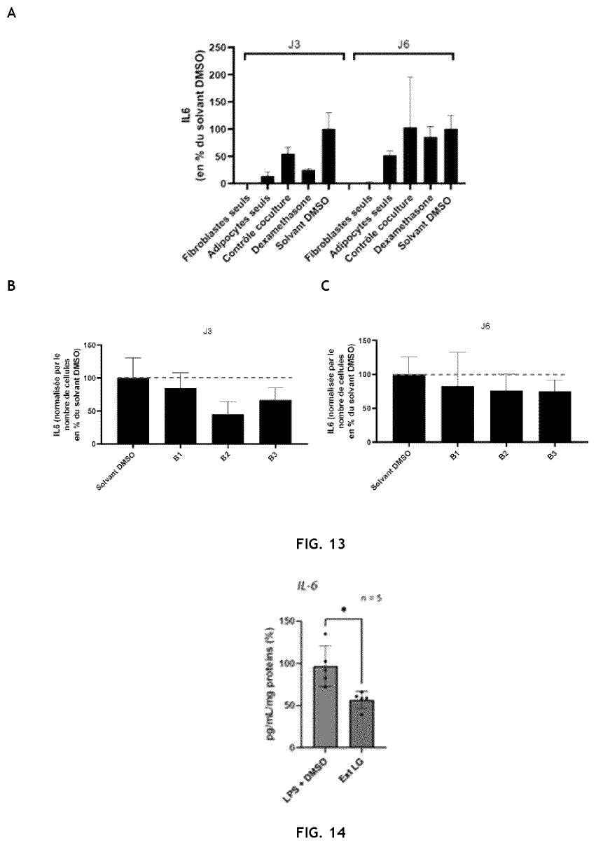

To assess the effect of the test element on inflammation, we assayed extracellular concentrations of secreted IL6. The results were normalized by the number of fibroblasts and represented as a percentage of the solvent condition DMSO (FIG. 13).

In the assays at day D3 and D6, fibroblasts alone do not secrete IL6 or hardly at all, while adipocytes alone do (14% at D3 and 52% at D6). It is when the two populations are brought into contact that secretions of the pro-inflammatory cytokine are increased. As expected at D3, with the anti-inflammatory reference (Dexamethasone), it is showed a significant decrease in secretions of the pro-inflammatory cytokine IL6 (25%). At D6, the effect of Dexamethasone is more nuanced as we only have a decreasing trend compared to the coculture control (85%). At D3, the solvent DMSO (100%) seems to increase IL6 secretions in the coculture compared to the coculture control (55%), this effect tends to disappear at D6. As the solvent induces changes in IL6 secretions, it is important to compare the conditions treated with lignin flower, compared to the DMSO solvent conditions. At D3, lignin flower induces a significant decrease in IL6 secretions, especially at doses 2 and 3 (45% and 67%). After 6 days of treatment, lignin flower tends to decrease pro-inflammatory cytokine secretions in the same proportions whatever the dose tested. This anti-inflammatory result of lignin flower in the decrease of IL6 secretions, was confirmed as illustrated in FIG. 14.

### Ames Test to Evaluate the Genotoxic Potential (According to OECD 471)

The purpose of the test is to investigate the possible genotoxic activity exerted by the tested substance in bacterial strains of Salmonella typhimurium, with and without metabolic activation with S9.

The Ames test allows for detecting the induction of point mutations in nucleotide bases, such as deletions, insertions, transversions and frameshift mutations by using modified Salmonella typhimurium strains. These strains carry a defective gene in the histidine operon making them auxotroph for this amino acid (mutants His-which require histidine in the culture medium for growth). The method guiding principle is based on the reverse mutation phenomenon by which bacteria exposed to a mutagenic substance may change back and become again prototroph concerning histidine (His+). The bacterial cells in growth phase are exposed to different concentrations of the test agent and mutagenic activity is determined by the capability of the test substance to induce a significant increase in the number of revertant (histidine-independent mutant, His+) in comparison to the spontaneous reversions occurring in the control cultures.

Some chemical agents are not directly mutagen but become so by following transformation and metabolic activation occurring in the organism by liver enzyme activity. To study this genotoxic effect, rat liver microsomal fraction (S9) has been added. The employ of S9 allows identifying substances that act as indirect mutagen.

### Ames Test Description

In this assay, 5 bacterial strains TA 1535, TA100, TA 102, TA 1537 and TA98 have been used and their characteristics are illustrated in Table 3. Each tester strains contains a different type of mutation in the histidine operon. TA 1535 and TA 100 strains are specific testers for mutagens causing base substitutions. The TA 102 strain is used to detect mutagens that require an intact excision repair system. The sensitivity of TA 100 and TA 102 is greatly enhanced by the introduction of an R factor, pKM101, which confers ampicillin resistance. Furthermore, the TA 102 strain contains the multicopy plasmid, pAQ1, which confers tetracycline resistance. The frameshift tester strains used are TA 1537 and TA 98. TA 98, like TA 100, is ampicillin resistant. All S. typhimurium strains carry, along with the defect in the histidine gene (His−), a deep rough (rfa) character, a mutation that causes partial loss of the lipopolysaccharide barrier that coats the surface of the bacteria and increases permeability to large molecules. Last, in all these strains, except TA 102, there is a deletion of a gene coding for the DNA excision repair system (uvrB), resulting in highly increased sensitivity in detecting many mutagens. For technical reason, the deletion excising the uvrB gene extends through the bio gene and, consequently, these bacteria also require biotin for growth.

TABLE 3
Characteristics of bacterial strains TA 1535, TA100, TA 102, TA 1537 and TA98.

| STRAINS | | POSITIVE CONTROLS | DOSAGE LEVELS |
|---|---|---|---|
| S. typhimurium TA 1535 | WITHOUT | sodium azide (NaN₃) in water | 1.5 μg/plate |
| S. typhimurium TA 1537 | S9 | 9-aminoacridine (9AA) in | 75 μg/plate |
| | | DMSO | |
| S. typhimurium TA 100 | | sodium azide (NaN₃) in water | 2.0 μg/plate |
| S. typhimurium TA 98 | | 4-nitroquinoline-N-oxide | 0.5 μg/plate |
| | | (4NQO) | |
| | | in DMSO | |
| S. typhimurium TA 102 | | Mitomycin C in water | 1.5 μg/plate |
| S. typhimurium TA 1535 | WITH | 2-aminoanthracene (2AAn) | 3.0 μg/plate |
| | S9 | in DMSO | |
| S. typhimurium TA 1537 | | 2-aminoanthracene (2AAn) | 3.0 μg/plate |
| | | in DMSO | |
| S. typhimurium TA 100 | | 2-aminoanthracene (2AAn) | 3.0 μg/plate |
| | | in DMSO | |
| S. typhimurium TA 98 | | 2-aminoanthracene (2AAn) | 3.0 μg/plate |
| | | in DMSO | |
| S. typhimurium TA 102 | | 2-aminoanthracene (2AAn) | 3.0 μg/plate |
| | | in DMSO | |

### Ames Test Procedure

The study was performed using the plate incorporation assay with and without S9 mix. S9 is composed by Aroclor 1254 induced rat liver supplemented with glucose-6-phosphate and NADP (Moltox®). The experiment was performed in duplicate Petri dishes containing basal medium and a further layer of medium containing histidine+biotin, the Salmonella typhimurium suspension, the sample to be tested at different concentrations and, if the case of metabolic activation, a 5% S9 mix. Dishes were incubated for 48 hours at 37° C. When the incubation time was over, a basal bacterial growth was achieved limited by the amount of histidine in the medium and in add the growth of revertant colonies (his+). In particular, the basic number of revertant colonies is steady, different for each strain, due to the spontaneous mutation rate of the bacterial strain. If an increase in the number of revertant colonies is observed, this is proportional to the tested sample concentration and to its mutagen capability. In each Ames test the following parameters are considered:
the negative control (or blank) represented by dishes used to detect the spontaneous revertant: bacteria that spontaneously, without any induction by the sample, revert to a normal condition. These dishes contain the solvents of control mutagens and the solvent of the tested substance (in this case dimethyl sulfoxide, DMSO and phosphate buffer PBS).
the positive control represented by the standard mutagens used to check the mutagen biological response of each strain, as described below.
   Direct Test: Mutagens without S9

S. typhimurium TA 1535: sodium azide (NaN3); S. typhimurium TA 1537:9-aminoacridine (9AA); S. typhimurium TA 100: sodium azide (NaN3); S. typhimurium TA 98:4-nitroquinoline-N-oxide (NQNO); S. typhimurium TA 102:4-nitroquinoline-N-oxide (NQNO).

The following tables report the average values as number of colony forming units (UFC)/plate for revertant obtained in the assay considering the average of 3 replicates for each dilution (Tables 4-6). According to the Ames tests, the sample of maple bark extract-lignin flower did not show any evidence of mutagenicity at the tested concentrations.

TABLE 4
TA 98 without S9

| | | | | | | Statistically |
|---|---|---|---|---|---|---|
| | | | | | | significant vs |
| | | | | | | solvent control |
| mg/plate | Replicate 1 | Replicate 2 | Replicate 3 | mean | ds | (p < 0.05) |
| 5.00 | 10 | 13 | 12 | 12 | 1.53 | No |
| 2.50 | 12 | 11 | 12 | 12 | 0.58 | No |
| 1.25 | 14 | 12 | 11 | 12 | 1.53 | No |
| 0.63 | 14 | 16 | 17 | 16 | 1.53 | No |
| 0.31 | 14 | 15 | 15 | 15 | 0.58 | No |
| PBS | 14 | 14 | 15 | 14 | 0.58 | — |
| DMSO | 14 | 15 | 15 | 15 | 0.58 | — |
| 4NQNO | 568 | 507 | 509 | 528 | 34.66 | yes |

TABLE 5
TA 98 with S9

| | | | | | | Statistically |
|---|---|---|---|---|---|---|
| | | | | | | significant vs |
| | | | | | | solvent control |
| mg/plate | Replicate 1 | Replicate 2 | Replicate 3 | mean | ds | (p < 0.05) |
| 5.00 | 14 | 14 | 15 | 14 | 0.58 | No |
| 2.50 | 14 | 16 | 14 | 15 | 1.15 | No |
| 1.25 | 11 | 14 | 14 | 13 | 1.73 | No |
| 0.63 | 14 | 16 | 16 | 15 | 1.15 | No |
| 0.31 | 17 | 18 | 18 | 18 | 0.58 | No |
| PBS | 21 | 23 | 17 | 20 | 3.06 | — |
| DMSO | 22 | 23 | 21 | 22 | 1.00 | — |
| 2AAn | 3353 | 3291 | 3316 | 3320 | 31.19 | yes |

TABLE 6
Skin irritation tests results

| | % mean cell viability (ds) | | |
|---|---|---|---|
| | 42 h treatment; | | |
| | 42 hours incubation | | Classification |
| Maple bark extract - | 97.94 | (2.17) | NI (Not irritant) |
| lignin flower | | | |
| SLS 5% | 0.93 | (0.03) | I (Irritant) |
| Positive Control (PC) | | | (UN-GHS |
| | | | Category 2) |
| Negative control (NC) | 100.00 | (3.45) | NI (Not irritant) |

### In Vitro Skin Irritation: Reconstructed Human Epidermis Test Method According to OECD 439

The purpose of this test is to evaluate if the tested material is, or is not a skin irritant, according to the method described in OECD 439. This method uses the human artificial skin model (EPISKIN™, EpiDerm™, Episkin™ RHE) to assess the skin irritation of chemical substances, mixtures and to properly label them to this respect if applicable.

The test is based on the evaluation of cell survival after the exposure to the substance through MTT assay and by comparison with epidermis treated with phosphate buffer only (negative control). The MTT method is a colorimetric assay that allows to determine the percentage of cells alive within an in vitro cultured tissue. This assay is based on the ability of the mitochondrial succinate dehydrogenase enzyme to metabolize the nitro-blue tetrazolium salt, giving a colored compound that can be measured by spectrophotometer reading. A reconstructed artificial human skin model comprising normal human epidermal keratinocytes, growing as an integrated three-dimensional cell culture model, perfectly mimicking the human skin in vitro. The model exhibits normal barrier functions (presence of a well-differentiated stratum corneum). It was supplied by Episkin (Lion, Batch 21-RHE-098) (FIG. 10).

The sample is tested as is, undiluted. The positive control SLS is dissolved at 5% in water. PBS alone has been used as negative control. 16 mg of the product have been applied on each epidermis unit (32 μL/cm²), in three replicates. The exposure has been carried out for 42 h at room temperature. At the end of the exposure period the product is removed with multiple washings with PBS and the tissue was further incubated at 37° C., 5% CO₂ for 42 h. After 42 h of the incubation, the viability assay is performed to evaluate the cell survival in the epidermis units.

Epidermis units are treated with 1 mg/ml MTT solution (3-[4,5-dimethylthiazol-2-yl]-2,5-diphenyl tetrazolium bromide) for 3 h at 37° C. The solution is then removed and replaced with isopropanol, with further 2 h incubation at room temperature. 2 aliquots of every sample are transferred to a 96 well plate for the reading. The absorbance is read at the wavelength of 570 nm with a colorimeter (Tecan model Infinite 200 PRO) equipped with a microplate reader. FIG. 10 represents a 3D epidermis model grown in vitro (SkinEthic™ RHE). Different layers of epidermis proliferation can be pointed out. At the surface, a well-differentiated stratum corneum is evident (red), over which the product to be tested is placed. Below the epidermis there is a semi-permeable membrane that communicates with the inferior well where the culture medium is placed.

All of the compositions and/or processes disclosed and claimed herein can be made and executed without undue experimentation in light of the present disclosure. While the compositions and processes of this disclosure have been described in terms of preferred embodiments, it will be apparent to those of skill in the art that variations may be applied to the compositions and/or processes and in the steps or in the sequence of steps of the processes described herein without departing from the concept, spirit and scope of the disclosure. More specifically, it will be apparent that certain agents which are chemically related may be substituted for the agents described herein while the same or similar results would be achieved. All such similar substitutes and modifications apparent to those skilled in the art are deemed to be within the spirit, scope and concept of the disclosure as defined by the appended claims.

While the present disclosure has been described in connection with specific embodiments thereof, it will be understood that it is capable of further modifications and this application is intended to cover any variations, uses, or adaptations including such departures from the present disclosure as come within known or customary practice within the art and as may be applied to the essential features hereinbefore set forth, and as follows in the scope of the appended claims.

1: A process for producing a depolymerized lignin comprising the steps of:
providing a solution comprising dissolved lignin;
adding an oxidation catalyst to the solution comprising dissolved lignin; and
heating the solution to produce the depolymerized lignin.

2: The process of claim 1, wherein the solution comprising dissolved lignin further comprises an alkali.

3: The process of claim 2, wherein the alkali comprises sodium hydroxide.

4: The process of claim 1, wherein the oxidation catalyst comprises sodium nitrite nanoparticles or carbon nanoparticles.

5: The process of claim 1, further comprising adding a co-catalyst to the solution comprising dissolved lignin.

6: The process of claim 5, wherein the co-catalyst comprises oxygen.

7: The process of claim 1, wherein the pH of the solution being heated ranges from about 12 to about 14.

8: The process of claim 1, wherein the heating comprises temperatures ranging from about 180° C. to about 200° C.

9: The process of claim 1, further comprising adding an alcohol to the depolymerized lignin, filtrating said depolymerized lignin and purifying the filtrated depolymerized lignin.

10: The process of claim 9, wherein the alcohol is isopropanol or ethanol.

11: The process of claim 1, wherein the depolymerized lignin product comprises at least one of vanillin at 15-25 wt. %; apocynin at 6-12 wt. %; acetosyringone at 12-15%; syringaldehyde at 13-30 wt. %; syringic acid at 16-24 wt. %; vanillic acid at 5-15 wt. %; and a combination thereof.

12-14. (canceled)

15: The composition use of claim 18, wherein the depolymerized lignin comprises at least one of vanillin at 15-25 wt. %; apocynin at 6-12 wt. %; acetosyringone at 12-15%; syringaldehyde at 13-30 wt. %; syringic acid at 16-24 wt. %; vanillic acid at 5-15 wt. %; and a combination thereof.

16-17. (canceled)

18: A cosmetic composition, a food additive, and/or an anti-inflammation composition comprising the depolymerized lignin produced by the process of claim 1 and a carrier.

19: The composition of claim 18, further comprising a of a plant, tree, fruit, or vegetable extract.

20: The composition of claim 19, wherein the plant, tree, fruit, or vegetable extract comprises a maple bark extract or a flower extract.
